(19) United States
(12) Patent Application Publication
(10) Pub. No.: US 2017/0082632 A1
(43) Pub. Date: Mar. 23, 2017

(54) KERATIN 17 AS A BIOMARKER FOR HEAD AND NECK CANCERS

(71) Applicant: THE RESEARCH FOUNDATION FOR THE STATE UNIVERSITY OF NEW YORK, Albany, NY (US)

(72) Inventors: Kenneth SHROYER, Setauket, NY (US); Michelle MO, Selden, NY (US); Annie SHROYER, Setauket, NY (US)

(73) Assignee: THE RESEARCH FOUNDATION FOR THE STATE UNIVERSITY OF NEW YORK, Albany, NY (US)

(21) Appl. No.: 15/311,611

(22) PCT Filed: May 15, 2015
(86) PCT No.: PCT/US15/30932

§ 371 Date: Nov. 16, 2016

Related U.S. Application Data
(60) Provisional application No. 61/994,492, filed on May 16, 2014.

(51) Int. Cl. G01N 33/574

(52) U.S. Cl. CPC G01N 33/57496; G01N 2333/4742; G01N 2800/52

(57) ABSTRACT

The current disclosure provides methods for detecting and analyzing KRT17 expression in a sample obtained from a subject. The current disclosure also pertains to methods and kits for identifying a mammalian subject with head and neck squamous cell carcinoma. The current disclosure further provides methods and kits for determining the likelihood of survival of a subject having head and neck squamous cell carcinoma.

### CROSS REFERENCE TO RELATED APPLICATIONS

This application claims priority from U.S. Provisional Application No. 61/994,492, filed May 16, 2014 the entire contents of which are incorporated herein by reference.

### INCORPORATION BY REFERENCE OF SEQUENCE LISTING

The Sequence Listing in the ASCII text file, named as 30915_SequenceListing.txt of 2 KB, created on Apr. 15, 2015, and submitted to the United States Patent and Trademark Office via EFS-Web, is incorporated herein by reference.

### FIELD OF THE DISCLOSURE

The current disclosure relates to a method of diagnosing squamous cell carcinomas (SCCs) of the head and neck that indicate the presence of squamous cell cancers in a subject. The current disclosure further provides methods for analyzing protein expression levels of cytokeratin 17 (KRT17 or keratin 17) in subjects in order to determine the presence of squamous cell cancer or the presence of a pre-cancerous lesion in a subject and the subject's survival rate and clinical outcome.

### BACKGROUND

Head and neck squamous cell carcinoma (HNSCC) is the sixth most common malignancy worldwide and is diagnosed in more than 600,000 individuals annually. Despite advances in treatment modalities, the prognosis of patients with HNSCC remains poor, with an average five-year survival rate of 40-50%. See, Bauman, J. E., et al. Current opinion in oncology (2012) Vol. 24, pp. 235-242; and Rothenberg, S. M., and Ellisen, L. W. The Journal of clinical investigation (2012) Vol. 122, pp. 1951-1957.

Currently, human papilloma virus (HPV) status is the only prognostic biomarker used in clinical practice as an indicator of survival-time from diagnosis of HNSCC, whereby HPV-positive tumors are associated with better survival in patients with HNSCCs. See, Dahlstrand, H., et al. Anticancer research (2008) Vol. 28, pp. 1133-1138. However, because approximately 75% of all HNSCCs are HPV-negative, no prognostic biomarkers are currently available for the vast majority of subjects having HNSCC. Thus, there is an unmet need to identify novel prognostic biomarkers that could be used in conjunction with HPV testing to better predict patient outcomes and survival time from initial diagnoses. For example, the identification of such prognostic biomarkers could potentially be used to determine optimal treatment regimens and to more accurately predict patient survival.

Cytokeratin 17 (KRT17), a member of the intermediate filament cytoskeleton family, is overexpressed in squamous cell carcinomas of the uterine and cervical mucosa and elevated KRT17 expression in cervical SCCs is predictive of poor clinical outcome in subjects diagnosed with cervical cancers. See Escobar-Hoyos, L. F., et al. Modern pathology (2014) Vol. 27(4), pp. 621-630. However, to date, no analysis of KRT17's role in the development and progression of HNSCC has been elucidated.

The current disclosure identifies and validates: (i) KRT17's utility as a diagnostic cancer biomarker; (ii) KRT17's utility as a prognostic biomarker of time-to-death for patients with HNSCCs; and (iii) KRT17's use in combination with HPV biomarkers to predict HNSCC patient survival time.

### SUMMARY OF THE DISCLOSURE

The current disclosure reveals that keratin 17 (KRT17) is a predictive biomarker for diagnosing head and neck cancers. Additionally, the data provided herein unexpectedly shows that KRT17 levels were significantly increased in subjects with well-differentiated squamous cell carcinoma of the tongue, tonsils or larynx, but absent or detected at low levels in normal mucosa or poorly differentiated squamous cell carcinoma (i.e., non-cancerous subject). Taken together, the current disclosure reveals that increased KRT17 expression is a critical event in the development and progression of HNSCC and that KRT17 staining can be measured as a diagnostic and prognostic indicator of HNSCC patient survival.

Therefore, in one aspect of the present disclosure an increased level of expression of KRT17 in subjects with squamous cell carcinoma of the head and neck is defined. In one embodiment, when an increased level of KRT17 expression, e.g., at least 5% of cells exhibit strong (2+) KRT17 staining, is detected in a sample obtained from a subject, the subject has HNSCC.

In another aspect of the present disclosure an increase in KRT17 protein expression has been correlated with a reduced incidence of survival in subjects diagnosed with HNSCC. In certain embodiments, an increase in KRT17 expression is detected in a sample, which is obtained from a subject having HNSCC, whereby such increase in KRT17 expression indicates a shorter survival time of such subject when compared to a subject having normal or reduced KRT17 expression levels. In a specific embodiment of the present disclosure, when an increased level of KRT17 expression, e.g., at least 85% of cells exhibit strong (2+) KRT17 staining, is detected in a sample obtained from a subject, the subject has a reduced likelihood of survival compared to a subject diagnosed with HNSCC that does not exhibit strong KRT17 staining in at least 85% of cells in a sample.

### BRIEF DESCRIPTION OF THE DRAWINGS

FIG. 1. KRT17 is overexpressed in lingual, tonsillar, and laryngeal SCCs. (a-c) Well-differentiated SCCs. (d-f) Moderately differentiated SCCs. (g-i) Poorly differentiated SCCs. (j-l) These data reveal a varying degree of KRT expression in each type of tumor (i.e., well differentiated, moderately differentiated and poorly differentiated) with no KRT17 expression exhibited in stromal elements. 100× magnification.

FIG. 2. KRT17 expression is heterogeneous between tumors. Proportion of cells with KRT17 strong (2+) intensity staining. Normal mucosa (2.9%±3.5%, n=34), lingual SCC (60.6±6.6%, n=39), tonsillar SCC (53.2±7.7%, n=25), and laryngeal SCC (50.9±6.8%, n=35). Values plotted as mean±SEM. All means of KRT17 expression in SCCs were significantly increased when compared to KRT17 expression levels in normal mucosa (p<0.05).

FIG. 3. KRT17 expression is reduced by grade and decreased expression in stage 2 versus stage 1 SCCs. Samples obtained from subjects were separated into grade and stage, and plotted based on the percentage of cells with strong 2+KRT17 staining intensity. (a) Grade—normal mucosa, i.e., control samples, (2.8%±0.5%, n=34), Grade 1 (74.6±6.9%, n=11), Grade 2 (51.6±5.3%, n=43), and Grade 3 (51.5±8.0%, n=22). (b) Stage —normal mucosa (2.8%±0.5%, n=36), Stage 1 (68.5%, ±9.0%, n=13), Stage 2 (39.3%±7.0%, n=25), Stage 3 (62.5±8.2%, n=15), Stage 4 (55.9±7.5%, n=22). All values were plotted as mean±SEM. An asterisk denotes p<0.05.

FIG. 4. KRT17 within tumors demonstrates different staining patterns, unrelated to anatomic site. (a) KRT17 staining at the periphery of infiltrative nests of tumor cells. (b) KRT17 checkerboard pattern. (c) KRT17 diffuse, uniform pattern. In some cases, the staining pattern was consistent through the tumor. In others, two or more patterns were observed. Images taken at 400× magnification.

FIG. 5. KRT17 is expressed in both low-grade squamous intraepithelial lesions and in high-grade squamous intraepithelial lesions. (a) KRT17 is overexpressed in LSIL (400× magnification) and in (b) HSIL (400× magnification). Diffuse staining was also rarely seen in benign squamous mucosa adjacent to tumor (panel a) independent of the level of KRT17 expression seen in the tumor cells. KRT17 staining in the respiratory mucosa was limited to basal and parabasal metaplastic cells.

FIG. 6. KRT17 expression pattern is maintained in nodal metastases. (a) KRT17 staining in a primary SCC and (b) in the corresponding lymph node metastasis. 200× magnification.

FIG. 7. HPV status of lingual, tonsillar, and laryngeal SCCs. HPV positive status for each site—27.6% of lingual SCCs (n=8/29), 42.9% of tonsillar SCCs (n=9/21), and 27.2% of laryngeal SCCs (n=6/22) were HPV positive.

FIG. 8. Kaplan-Meier survival curves of HNSCC patients; high versus low KRT17 expression. HNSCC cases from all sites (tongue, tonsil, larynx) (n=78). High (i.e., Strong (2+)) KRT17 expression was associated with decreased survival in SCCs of all sites as a single group (p=0.006).

FIG. 9. Kaplan-Meier survival curves of lingual and tonsillar SCC patients; high versus low KRT17 expression. (a) Lingual SCCs (n=29), (b) Tonsillar SCCs (n=24). High (i.e., Strong (2+)) KRT17 expression was associated with decreased survival in lingual SCCs (p=0.0061) and tonsillar SCCs (p=0.0235).

Table 1. Patient Characteristics. The population for survival analysis consisted of primary or recurrent tumor samples (n=78). Numbers in parentheses represent confidence intervals.

### DETAILED DESCRIPTION OF THE DISCLOSURE

Currently, an HPV-positive status is the only prognostic biomarker used by clinicians to predict survival-time for subjects diagnosed with HNSCCs. However, whether or not an HPV-positive status correlates with a certain clinical outcome is unclear. See Rautava, J., et al. Journal of clinical virology (2012) Vol. 53, pp. 116-120; Rosenquist, K., et al. Acta oto-laryngologica (2007) Vol. 127, pp. 980-987; and Simonato, L. E., et al. Journal of oral pathology & medicine (2008) Vol. 37, pp. 593-598. As such, the identification of diagnostic and/or prognostic biomarkers for HNSCCs is crucial in order to develop molecular screens, methods for diagnosing subjects with HNSCC and creating diagnostic or prognostic kits to determine the appropriate treatment regimens and improve both treatment strategies of HNSCCs and patient outcomes.

### DEFINITIONS

The term “cytokeratin 17” or “keratin17” or “KRT17” as used herein refers to a 432 amino acid polypeptide as set forth in accession number CAA79626.1, which is transcribed from the keratin 17 gene located on human chromosome 17 (17q21.1) as set forth in RefSeq. NC_000017.11 and homologs thereof. In certain non-limiting examples of homologs of the KRT17 gene, the keratin 17 coding region is conserved, as in Rhesus monkey, chimpanzee, dog, bovine, mouse, and rat.

The term “peptide”, “polypeptide” or “protein” as used in the current disclosure refers to a linear series of amino acid residues linked to one another by peptide bonds between the alpha-amino and carboxy groups of adjacent amino acid residues. In one embodiment the protein is keratin 17 (KRT17).

The term “larynx” as used herein shall mean the tubular structure connecting the trachea and lungs, which facilitates the passage of air into the lungs. More specifically, the larynx is composed of an external skeleton of cartilage plates that prevents collapse, such plates are interconnected by membranes and muscle tissue. The larynx has three distinct regions (i.e., supraglottic, glottis, and subglottic), which are clinically different in terms of local spread and metastatic potential and gene expression.

The term “pharynx” as used herein shall mean a muscular tube that passes downward through the neck and facilitates the passage of air to the larynx and food to the esophagus and then stomach. An upper portion of the pharynx contains an auditory canal, which opens onto the upper part of the pharynx. Moreover, the walls of the pharynx are composed of fascia and muscle layers, which are lined with a mucous membrane. The pharynx is composed of three distinct regions the nasopharynx, which is located behind the nose; the oropharynx that is behind the mouth; and the laryngopharynx, which is located behind the larynx.

The term “tonsil(s)” as used herein means a ring of lymphoid tissue surrounding the upper part of the pharynx. Tonsils consist of three regions: the lingual tonsil in the posterior part of the tongue; the palatine tonsils and the pharyngeal tonsils.

The term “squamous cell cancers of the head and neck”, “head and neck cancer” or “head and neck squamous cell carcinomas” (collectively, “HNSCC”) as used herein shall mean neoplastic lesions or tissues located within the oral cavity or mouth of a subject. The oral cavity includes: (i) the vestibule, which is the space between the lips and innermost surface of the cheeks and teeth and gums; (ii) the mouth proper, which refers to the tissue and structures that are internal to the teeth; (iii) the tonsils; (iv) the pharynx; and (v) the larynx. More specifically, the oral cavity refers to the entire contents of the cheeks, gums, teeth, tongue, larynx, pharynx, tonsils and palate.

The term “lingual squamous cell carcinoma” or “lingual cancer” as used herein means neoplastic tissues located on or near the tongue within the oral cavity.

The term “tonsillar squamous cell carcinoma” as used herein shall mean neoplastic or malignant lymphoid tissue of the tonsil or tonsils located within the oral cavity of a subject.

The term “laryngeal squamous cell carcinoma” or “squamous cell carcinoma of the larynx” as used herein shall mean neoplastic or malignant tissue of the supraglottic, glottis, and/or subglottic regions of the larynx within the oral cavity of a subject.

The phrase “subject” or “patient” as used herein refers to any mammal. In one embodiment the subject is a candidate for cancer diagnosis or an individual with HNSCC or the presence of a pre-cancerous lesion thereof. The methods of the current disclosure can be practiced on any mammalian subject that has a risk of developing HNSCC. Particularly, the methods described herein are most useful when practiced on humans. In certain preferred embodiments, a subject is an individual previously diagnosed with HNSCC, where by such subject is in need of an anticancer therapy.

A “biological sample,” “sample” or “samples” to be used in the disclosure can be obtained in any manner known to a skilled artisan. Samples can be derived from any part of a subject's oral cavity including, but not limited to, mucosal membranes, tissue, lymph node or a combination thereof. In certain embodiments, the sample is a formalin-fixed paraffin-embedded tissue isolated from a subject. Formalin-fixed paraffin-embedded tissue is useful in the methods of the current disclosure because formalin fixation and paraffin embedding is universally used for the histological preservation and diagnosis of clinical tissue specimens, and formalin-fixed paraffin-embedded tissues are more readily available in large amounts than fresh or frozen tissues. In yet other embodiments the sample is a tissue biopsy, fresh tissue or live tissue extracted from a subject.

Conversely, a “control sample” or “normal sample” as used herein is a sample which does not contain cancerous cells (e.g., benign tissue components including, but not limited to, normal squamous mucosa, oropharyngeal mucosa cells, lymphocytes, and other benign mucosal tissue components); a sample which does not exhibit elevated KRT17 expression levels, e.g., samples from benign or cancerous tissues. Non-limiting examples of control samples for use in the current disclosure include, non-cancerous tissue extracts, surgical margins extracted from the subject, isolated cells known to have normal or reduced KRT17 expression levels, obtained from the subject under examination or other healthy individuals. In one specific embodiment, the control sample of the present disclosure is benign mucosal tissue. In yet another embodiment, the control sample exhibits KRT17 expression in less than 5% of cells. In one embodiment of the current disclosure, the amount KRT17 in a sample is compared to either a standard amount of KRT17 present in a normal cell or a non-cancerous cell, or to the amount of KRT17 in a control sample or database. In a specific embodiment, a control sample is a tissue sample that has no recognizable staining for KRT17 (i.e., 0 pathology score) when viewed microscopically. The comparison can be done by any method known to a skilled artisan.

The term “increase” or “greater” or “elevated” means at least more than the relative amount of an entity identified (such as KRT17 expression), measured or analyzed in a control sample. Non-limiting examples include, but are not limited to, a 5%, 5-10%, 10-20% increase over that of a control sample, or at least 20%, 30%, 40%, 50%, 60%, 70%, 80%, 85% 90%, 100%, 200% or greater increase over that of a control sample, or at least a 0.25 fold, 0.5 fold, 1 fold, 1.5 fold, 2 fold, 3 fold, 4 fold, 5 fold, 10 fold, 11 fold or greater, increase relative to the entity being analyzing in the control sample. In certain embodiments, an increase in KRT17 expression is exhibited by an increase in KRT17 staining intensity or the number of KRT17 positive cells in a sample.

The term “decrease”, “reduced” or “reduction” means at least lesser than the relative amount of an entity identified (e.g., KRT17 expression), measured or analyzed in a control sample. Non-limiting examples, include but are not limited to, 1%, 2%, 3%, 4%, 5%, 5-10%, 10-20% decrease compared to that of a control sample, or at least 5%, 10%, 20%, 30%, 40%, 50%, 60%, 70%, 80%, 90%, 100%, 200% or greater decrease when compared to that of a control sample, or at least a 0.25 fold, 0.5 fold, 1 fold, 1.5 fold, 2 fold, 3 fold, 4 fold, 5 fold, 10 fold, 11 fold or greater, decrease relative to the entity being analyzing in the control sample.

The term “strong staining” “2+staining” or “strong KRT17 staining” as used herein shall mean a pathologist performed semi-quantitative analysis, by microscopic examination of a sample that identifies the percentage of cells stained by a KRT17 antibody (i.e., percentage of KRT17 positive cells) within the stained tissue, whereby dark-stained-tumor cells were scored as 2+ or strongly stained cells. In certain embodiments, strongly stained cells or samples exhibiting strong staining are used to determine whether a subject has HNSCC. In a specific embodiment, when at least 5% of the cells in a sample exhibit strong (i.e., 2+staining) KRT17 staining, the subject from which the sample was obtained will be diagnosed with HNSCC. In yet another embodiment, the strongly stained cells or samples exhibiting strong KRT17 staining are used to determine the survival time of a subject. For example, when at least 85% of the cells in a sample exhibit strong KRT17 staining, the subject from which the sample was obtained will have a reduced survival time.

An “increased level of KRT17 expression” or “high level of KRT17 expression” as used in the current disclosure shall mean an increase in the amount of KRT17 protein expression present in a cell, organism or sample as compared to a control or normal level of KRT17 expression in a subject or sample obtained therefrom. In an embodiment of the present disclosure, an increase in KRT17 expression is shown by strong (i.e., 2+) KRT17 staining intensity in a sample. In certain embodiments, an increased level of KRT17 expression, which is indicative of the presence of HNSCC in a subject, is when least 5% of the cells in a sample exhibit KRT17 expression. In yet another embodiment, an increased level of KRT17 expression, which is indicative of a reduced likelihood of survival compared to a subject diagnosed with HNSCC that does not exhibit strong KRT17 staining, is when least 85% of the cells in a sample exhibit KRT17 expression.

A “low level of KRT17 expression” as used in the current disclosure shall mean the presence of KRT17 staining in less than 5% of the cells present in a sample. In certain embodiments a low level of KRT17 expression is an amount of KRT17 protein expression present in a control sample or the level of KRT17 expression in benign mucosal tissue obtained from a subject. In a specific embodiment, a low level of KRT17 expression is when less than 85% of cells present in a sample exhibit strong KRT17 staining. More specifically, a low level of KRT17 expression is when less than 5% of cells present in a sample exhibit strong KRT17 staining.

### Diagnostic Methods

In certain aspects of the present disclosure KRT17 has been identified and validated as a novel biomarker for the diagnosis of head and neck squamous cell carcinomas. In certain embodiments, the present disclosure provides a method of diagnosing HNSCC in a subject based on detecting increased KRT17 expression.

A biological sample is obtained from the subject in question. The biological sample that can be used in accordance with the present disclosure may be collected by a variety of means. Non-limiting examples include, by surgery, by paracentesis needle for tissue collection, or by collection of body fluid, secretion from a gland, blood extract or urine. In some embodiments, the sample obtained from a subject is used directly without any preliminary treatments or processing, such as fractionation or DNA extraction. In other embodiments, the sample is processed such that DNA or proteins can be extracted or enriched from the sample before detecting expression levels. Methods of extracting proteins or DNA from biological sample are well known in the art, and may be performed using, for example, DNA extraction can occur via phenol/chloroform, ethanol, or commercially available DNA extraction reagents; similarly, proteins may be extracted by using a TCA-acetone), phenol, or multi-detergents in a chaotrope solution and examined during gel electrophoresis and analyzed by mass spectrometry, proteins may also be extracted and isolated using GST-pull down techniques that are well known in the art.

In certain specific embodiments, protein expression in a sample is analyzed by immunohistochemistry, whereby immunohistochemical staining enables the determination of the presence of specific peptide (e.g., protein) within cells of a tissue. More specifically, samples may be obtained from a subject, processed onto glass slides and stained using indirect avidin-biotin based immunoperoxidase methods. The samples are then incubated with KRT17 antibodies and analyzed via microscopy for KRT17 expression.

In a preferred embodiment, immunohistochemical staining was used to visualize KRT17 expression, by using a primary antibody specific against KRT17, followed by detection using an enzyme-linked antibody. The enzyme causes a chemical reaction in the tissue adjacent to the site of antibody binding that results in the oxidative reaction of 3,3′ diaminobenzidine (DAB). Oxidized DAB produces a dark (brown) deposition in cells, indicating the presence of KRT17.

In certain embodiments, tissue samples from a subject was stained using above immunohistochemical process and the amount of KRT17 expression in each case was quantified by a pathologist by microscopic examination of each sample to define the percentage of stained tumor cells (i.e., percentage of KRT17 positive) within the stained tissue. Dark-stained-tumor cells were scored as 2+, while the light-stained-tumor cells were scored as 1+. Cells with no staining were scored as 0. Only the percentage of total tumor cells that had dark-brown staining (2+) were considered for the statistical analyses on diagnostic and/or prognostic performance of KRT17. Furthermore, to determine the low and high KRT17 cases for the diagnostic and/or prognostic performance analyses, threshold percentages were defined based on an optimal positive likelihood ratio in all cases.

In specific embodiments, two thresholds for KRT17 were established: 1) diagnostic threshold for KRT17 positive cells≧5%, i.e., samples showing strong KRT17 staining in at least 5% of cells were indicative of HNSCC, and 2) prognostic threshold for KRT17 positive cells≧85%, i.e., samples showing strong KRT17 staining in at least 85% of cells were indicative of HNSCC. These two thresholds were used in all analyses performed the results of which are provided herein.

In one embodiment, KRT17 expression measured from the subject or sample in question is compared to a control value in order to determine whether or not HNSCC exists in the subject. In some embodiments the KRT17 staining of a sample will be compared to that of a control sample, obtained from non-cancerous or benign tissue. For example, an alteration in KRT17 expression as evidenced by an increase in KRT17 expression relative to a control value indicates that the subject has HNSCC. Alternatively, when the level of KRT17 is decreased or equal to a control value, it can be determined that said subject does not have prostate cancer. A control value can be a pre-determine value or can be determined from a control sample side by side with the sample obtained from the subject in question.

In yet another embodiment, the control value is established from a control sample obtained from benign tissue including, but not limited to, normal oropharyngeal mucosa or benign mucosal tissue. That is, the level of KRT17 expression in a test sample is compared to that of a sample obtained from benign tissue (i.e., control sample) including, but not limited to, benign squamous mucosa. If the amount of KRT17 expression in the test sample is greater than the amount of KRT17 expression in the control sample, then the subject is diagnosed as having HNSCC.

As demonstrated herein, immunohistochemical analysis confirmed that KRT17 expression was significantly increased in head and neck squamous cell carcinoma when compared to normal oropharyngeal mucosa or benign squamous mucosa (i.e., control samples). In certain embodiments, the significant increase in keratin 17 expression that corresponds with a diagnosis of head and neck squamous cell carcinoma is exemplified by strong (2+) KRT17 staining in ≧5%, or between 5% and 10% of cells in a sample, inclusive. In other embodiments, strong KRT17 expression in at least 10% of the cells in a sample corresponds with HNSCC. In a specific embodiment, the KRT17 expression value that corresponds with a diagnosis of head and neck squamous cell carcinoma is exemplified by strong KRT17 staining (i.e., 2+KRT17 staining) in 5% to 100% of the cells in a sample.

In certain embodiments, a KRT17 expression value that corresponds with the diagnosis of lingual SCC in a subject is 2+KRT17 staining in between 50% and 70% of cells in a sample, 54% and 67% of cells in a sample or in about 54, 55, 56, 57, 58, 59, 60, 61, 62, 63, 64, 65, 66, or 67 percent of cells in a sample.

In another embodiment, a KRT17 expression value that corresponds with the diagnosis of tonsillar SCC in a subject is 2+KRT17 staining in between 45% and 65% of cells in a sample, 45% and 61% of cells in a sample or in about 44, 45, 46, 47, 48, 49, 50, 51, 52, 53, 54, 55, 56, 57, 58, 59, 60 or 61 percent of cells in a sample.

In another embodiment, a KRT17 expression value that corresponds with the diagnosis of laryngeal SCC in a subject is 2+KRT17 staining in between 40% and 60% of cells in a sample, 44% and 58% of cells in a sample or in about 44, 45, 46, 47, 48, 49, 50, 51, 52, 53, 54, 55, 56, 57, 58, 59, or 60 percent of cells in a sample.

In certain aspects of the present disclosure the level of KRT17 expression is used as a basis to determine the severity of HNSCC in the subject. In certain embodiments KRT17 expression levels can be used to determine the grade and/or stage of a HNSCC in a sample.

According to the current disclosure, the extent of the increase in KRT17 expression relative to a control correlates with the grade of the cancer. In a non-limiting example the association between Gleason score and KRT17 expression was determined, whereby the average of HNSCC grade % (outcome variable) was calculated for a subject and a standard descriptive summary was then performed for this grade % average stratified by three categories. Kruskal-Wallis nonparametric test or Wilcoxon rank-sum test was used to evaluate the difference among three or between two of the three categories, respectively.

In one embodiment of the present disclosure a Grade 1 HNSCC is a sample containing 2+KRT17 staining in greater than 65% of cells in a sample. In yet another embodiment, a Grade 1 HNSCC is a sample containing 2+KRT17 staining in between 65% and 80% of cells in a sample, inclusive. In another embodiment, a Grade 1 HNSCC is a sample containing 2+KRT17 staining in about 66, 67, 68, 69, 70, 71, 72, 73, 74, 75, 76, 77, 78, 79, 80, 81, 82 or 83% of cells in a sample.

In yet another embodiment of the present disclosure a Grade 2-3 HNSCC is a sample containing 2+KRT17 staining in greater than 50% of cells in a sample. In yet another embodiment, a Grade 2-3 HNSCC is a sample containing 2+KRT17 staining in between 43% and 60% of cells in a sample, inclusive. In another embodiment, a Grade 2-3 HNSCC is a sample containing 2+KRT17 staining in about 43, 44, 45, 46, 47, 48, 49, 50, 51, 52, 53, 54, 55, 56, 57, 58, 59, 60 and 61% of cells in a sample.

In one embodiment of the present disclosure, the present disclosure a Stage 1 HNSCC is identified as a sample containing 2+KRT17 staining in greater than 59% of cells in a sample. In yet another embodiment, a Stage 1 HNSCC is a sample containing 2+KRT17 staining in between 59% and 78% of cells in a sample, inclusive. In another embodiment, a Stage 1 HNSCC is a sample containing 2+KRT17 staining in about 59, 60, 61, 62, 63, 64, 65, 66, 67, 68, 69, 70, 71, 72, 73, 74, 75, 76, 77, or 78% of cells in a sample.

Unexpetedly, a Stage 2-3 HNSCC exhibited a reduction in the amount of 2+KRT17 staining, when compared to Stage 1 HNSCC. Therefore, a Stage 2-3 HNSCC is determined by strong, 2+KRT17 staining in between 32% and 70% of cells in a sample. In yet another embodiment, a Stage 2-3 HNSCC is a sample containing 2+KRT17 staining in between 39% and 62% of cells in a sample, inclusive. In another embodiment, a Stage 2-3 HNSCC is a sample containing 2+KRT17 staining in about 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or 71% of cells in a sample.

In yet another embodiment of the present disclosure a Stage 4 HNSCC is distinguished by identifying a sample containing 2+KRT17 staining in greater than 52% of cells in such sample. In yet another embodiment, a Stage 4 HNSCC includes a sample containing 2+KRT17 staining in between 52% and 63% of cells in a sample, inclusive. In another embodiment, a Stage 4 HNSCC includes is a sample containing 2+KRT17 staining in about 52, 53, 54, 55, 56, 57, 58, 59, 60, 61, 62, or 63% of cells in a sample.

In a specific embodiment, Stage 1 HNSCC is exhibited in sample containing 2+KRT17 staining in about 68% of cells in a sample; a Stage 2-3 HNSCC is exhibited in sample containing 2+KRT17 staining in about 40% of cells in a sample; and a Stage 4 HNSCC is exhibited in sample containing 2+KRT17 staining in about 56% of cells in a sample. In a preferred embodiment, Stage 1 HNSCC is exhibited in sample containing 2+KRT17 staining in between 60%-77% of cells in a sample; a Stage 2-3 HNSCC is determined by 2+KRT17 staining in between 32%-48% of cells in a sample; and a Stage 4 HNSCC is exhibited in sample containing 2+KRT17 staining in between 49%-59% of cells in a sample.

In yet another embodiment, the level of KRT17 expression of a subject is used to diagnose a subject as having HNSCC and to further determine the severity of HNSCC in the subject. For example, the level of KRT17 expression in a test sample is obtained and subsequently compared to that of a sample obtained from benign tissue (i.e., control sample) including, but not limited to, benign squamous mucosa. If the amount of KRT17 expression in the test sample is significantly greater than the amount of KRT17 expression in the control sample, then the subject is diagnosed as having HNSCC. In certain embodiments, the significant increase in KRT17 expression that corresponds with a diagnosis of head and neck squamous cell carcinoma in a subject is exemplified by strong (2+) KRT17 staining in 5%, or between 5% and 10% of cells in a sample, inclusive. In a specific embodiment, strong KRT17 expression in at least 10% of the cells in a sample corresponds with a diagnosis of HNSCC.

After such diagnosis is made, the amount of strong KRT17 staining in a subject can be further analyzed to determine the severity of HNSCC in the subject, as set forth above. Certain non-limiting examples of such a determination includes identifying: a Grade 1 HNSCC as a sample containing 2+KRT17 staining in greater than 65% of cells in the sample; and/or a Grade 2-3 HNSCC in a sample having 2+KRT17 staining in between 32 and 48% of cells in a sample. A Stage 4 HNSCC is distinguished by identifying a sample containing 2+KRT17 staining in between 49%-59% of cells in such sample. Similarly, disease severity can be determined by using the level of 2+KRT17 staining in a sample to elucidate the stage of HNSCC in a subject. For example, Stage 1 HNSCC is exhibited in sample containing 2+KRT17 staining in between 60%-77% of cells in a sample; a Stage 2-3 HNSCC is determined by 2+KRT17 staining in between 32%-48% of cells in a sample; and a Stage 4 HNSCC is exhibited in sample containing 2+KRT17 staining in between 49%-59% of cells in a sample.

### Prognostic Methods

In one aspect of the present disclosure KRT17 expression is used as a prognostic biomarker of poor survival outcome for subjects having HNSCCs, e.g., tonsillar and lingual squamous cell carcinomas. Generally, KRT17 has been identified as a novel prognostic biomarker of patient survival (independent of HPV status, grade, and stage) for patients with lingual and tonsillar SCCs. More specifically, the absence of strong (2+) KRT17 expression in samples obtained from subjects diagnosed with HNSCC resulted in a longer survival rate, while increased KRT17 expression levels (i.e., strong, 2+KRT17 expression) in patients diagnosed with head and neck cancer resulted in shorter survival times for the subject. In certain embodiments, the methods of the present disclosure show that subjects exhibiting strong KRT17-expression in at least 85% of in lingual or tonsillar SCC cells were 3.8 times or 5.9 times, respectively, less likely to survive as long as subjects exhibiting low KRT17 expression in lingual or tonsillar SCCs samples.

In certain embodiments the level of KRT17 expression in a sample is determined by determining an ImageJ score or a PathSQ score for a subset of patients and choosing an appropriate level of KRT17 expression according to the lowest Akaike's information criteria in view of a Cox proportional-hazard regression model. In other embodiments, a low level of KRT17 expression is exemplified by the presence of strong KRT17 staining in less than 20% of the cells present in a sample. In a specific embodiment, a low level of KRT17 expression is exemplified by the presence of strong KRT17 staining in less than 85% of the cells in a sample. In another embodiment, a high level of KRT17 expression is exemplified by the presence of strong (2+) KRT17 staining in at least 85% of cells in a sample.

In an embodiment of the current disclosure high KRT17 expression levels are associated with poor survival of subjects having lingual (hazard Ratio=3.794, p=0.0061) or tonsillar (hazard Ratio=5.921, p=0.0235) squamous cell carcinoma. Thus, increased expression of KRT17 in a subject indicates that the subject will have a reduced likelihood of survival compared to a subject diagnosed with HNSCC that does not have an increase in KRT17 expression or exhibits no KRT17 expression.

In certain specific embodiments five-year survival rates of head and neck squamous cell carcinoma patients with low KRT17 expression were about 50%. Conversely, five-year survival rates of head and neck squamous cell carcinoma patients with high KRT17 expression were about 25%. In a specific embodiment, subjects exhibiting a strong level of KRT17 expression in at least 85% of cells in a sample had a five-year overall survival rate of 22% from initial diagnosis of HNSCC.

A similar trend was observed at the 10-year survival rates of head and neck squamous cell carcinoma patients, whereby ten-year survival rates of head and neck squamous cell carcinoma patients with low KRT17 expression were estimated at 45%, while no subjects diagnosed with head and neck squamous cell carcinoma having high KRT17 expression survived ten years from initial diagnosis. Taken together, the methods provided herein clearly show that KRT17 expression can be used as a prognostic biomarker of survival outcome for subjects having HNSCCs, e.g., tonsillar and lingual squamous cell carcinomas and to project time-to-death of a subject having HNSCC.

TABLE 1
Time to death analysis of subjects in view of tumor type, subject risk
factors, and whether or not chemotherapy was provided to each subject.

| | | | | | | Did Not | | |
|---|---|---|---|---|---|---|---|---|
| | | All | | Survived | | Survive | | |
| | | Freq | % | Freq | % | Freq | % | P Value |
| Total | | 78 | 100 | 34 | 43.59% | 44 | 56.41% | |
| Tissue | larynx | 25 | 32.05% | 11 | 32.35% | 14 | 31.82% | 0.947 |
| Location | tongue | 29 | 37.18% | 12 | 35.29% | 17 | 38.64% | |
| | tonsil | 24 | 30.77% | 11 | 32.35% | 13 | 29.55% | |
| Chemo | No | 45 | 57.69% | 23 | 67.65% | 22 | 50.00% | 0.118 |
| | Yes | 33 | 42.31% | 11 | 32.35% | 22 | 50.00% | |
| Radiation | No | 20 | 25.64% | 13 | 38.24% | 7 | 15.91% | 0.025 |
| | Yes | 58 | 74.36% | 21 | 61.76% | 37 | 84.09% | |
| Stage | 1 | 14 | 17.95% | 9 | 26.47% | 5 | 11.90% | 0.245 |
| | 2 | 25 | 32.05% | 11 | 32.35% | 14 | 33.33% | |
| | 3 | 15 | 19.23% | 4 | 11.76% | 11 | 26.19% | |
| | 4 | 22 | 28.21% | 10 | 29.41% | 12 | 28.57% | |
| HPV | No | 48 | 70.59% | 22 | 75.86% | 26 | 66.67% | 0.411 |
| | Yes | 20 | 29.41% | 7 | 24.14% | 13 | 33.33% | |
| Age | Mean | 60.14 | | 59.09 | | | | 0.072 |
| | | (57.29, | | (54.95, | | | | |
| | | 62.99) | | 63.23) | | | | |
| | Median | 59.5 | | 59.5 | | | | |
| | | (50, | | (51, | | | | |
| | | 68) | | 66) | | | | |
| KRT17 | Mean | 53.49 | | 45.59 | | | 59.52 | |
| | | (45.80, | | (33.34, | | | (49.78, | |
| | | 61.1) | | 57.84) | | | 69.26) | |
| | Median | 52.5 | | 50 | | | 60 | 0.05 |
| | | (20, | | (65, | | | (60, | |
| | | 68) | | 80) | | | 70.5) | |

### EXAMPLES

### Example 1

### Materials and Methods

Case Selection.

A total of 25 laryngeal SCCs, 29 lingual SCCs, and 24 tonsillar SCCs were selected from the archival Pathology collections of the Biobank at Stony Brook University Medical Center. Ten laryngeal, 9 lingual, and 15 tonsillar non-cancerous tissue specimens (i.e., control samples) were also selected from the archival pathology collections. Additionally, nodal metastases representative of all three anatomic regions were concurrently selected (n=6). Representative sections from each case were selected by a pathologist and grading was performed on a three-point scale (Grade 1-3).

Immunohistochemical Staining.

Immunohistochemical staining was performed on paraffin-embedded tissue samples obtained from subjects. Specifically, formalin-fixed, paraffin-embedded tissue sections obtained from subjects were marked on glass slides. After incubation at 60° C. for 1 h, tissue microarray slides were deparaffinized in xylene and rehydrated using graded alcohols. Antigen retrieval was performed in citrate buffer (20 mmol, pH 6.0) at 120° C. for 10 minutes in a decloaking chamber. Endogenous peroxidase was blocked by applying 3% hydrogen peroxide for 5 minutes. Sections were subsequently blocked in 5% horse serum. Sections were incubated with mouse monoclonal-[E3] anti-human KRT17 antibody (ab75123, Abcam, Cambridge, Mass., USA) at 4° C. overnight. After incubation with the primary antibody, slides were processed by an indirect avidin-biotin-based immunoperoxidase method using biotinylated horse secondary antibodies (R.T.U. Vectastain Universal Elite ABC kit; Vector Laboratories, Burlingame, Calif., USA), developed in 3,3′ diaminobenzidine (DAB) (K3468, Dako, Carpentaria, Calif., USA), and counter-stained with hematoxylin. Negative controls were performed on all cases using an equivalent concentration of a subclass-matched mouse immunoglobulin, generated against unrelated antigens (mouse IgG, BD PharMingen, San Diego, Calif.) in place of primary antibody. Staining was developed using 3,3′-diaminobenzidine (DakoCytomation, Carpentaria, Calif., USA) and slides were counterstained with hematoxylin. Slides were scored using 1 representative histologic section from each tissue specimen by a manual semi-quantitative scoring system based on the proportion of tumor cells with strong (2+) staining.

HPV Genotyping.

DNA was extracted from tissue blocks using the QIAamp DNA FFPE Tissue kit (Qiagen, Valencia, Calif.), with the following modifications: a 48-hour Proteinase K digestion and addition of RNA carrier prior to elution. HPV Types 16 and 18 were detected by PCR using HPV Type 16 forward primer 5′-CGCACAAAACGT GCATCGGCTACC-3′ (SEQ ID NO. 1) and reverse primer 5′-TGGGAGGCCTTGTTCCCAATGGA-3 (SEQ ID NO. 2); and HPV Type 18 forward primer 5′-AACAGTCCATTAGGGGAGCGGCTGGA-3′ (SEQ ID NO. 3) and reverse primer 5′-TGCCGCCATGTTCGCCATTTG-3′ (SEQ ID NO. 4). Beta-globin was detected and amplified by PCR for use as an internal control using forward primer: 5′-CAACTTCATCCACGTTCACC-3′ (SEQ ID NO. 5) and reverse primer: 5′-GAAGAGCCAAGGACAGGTAC-3′ (SEQ ID NO. 6). All PCRs were performed using the HotStar® Taq Plus Master Mix PCR Kit (Qiagen, Valencia, Calif.) according to the manufacturer's instructions. More specifically, the following cycling protocol was used: incubation at 95° C. for 15 min, followed by 40 cycles of denaturation for 30 seconds at 94° C., 1.5 min of annealing at 59° C. for beta-globin and 70° C. for HPV Type 16/18, and 1 min of elongation at 72° C. The last cycle was followed by a final extension step of 2 min at 72° C. Amplification was performed in a C1000 Touch Thermal Cycler, (Biorad Laboratories, Hercules, Calif.). For all reactions, positive controls (SiHa cells for HPV 16 and HeLa cells for HPV 18) were used. Normal placental tissue was used as negative controls to monitor for primer-specific annealing and potential contamination. For all tissue specimens that were HPV positive (n=23), PCR products were extracted and purified using the ExoSAP-IT Kit (Cleveland, Ohio, Affymetrix) then analyzed and sequenced by the Genomics Core Facility at Stony Brook University using an Applied Biosystems 3730 (48 cm capillary array) DNA analyzer according to manufacturers protocol. Six out of the 23 HPV positive samples were neither HPV16 nor HPV18 positive.

Statistical Analysis: Determining the Diagnostic Properties of KRT17.

A small subgroup of match-paired specimens were selected from the larger sample, with subjects having both cancerous and non-cancerous control tissues examined (n=21 patients, 42 specimens). A Wilcoxon signed rank-sum test was used to determine if there was a significant difference (p≦0.05) in KRT17 expression between the cancerous and non-cancerous tissue pairs for each subject. Upon detecting a significant difference (p≦0.05), a cut-off point for KRT17 expression was developed to identify cancer versus non-cancer specimens within the matched-paired dataset by creating receiver operating curves and using the area under the curve to evaluate KRT17 biomarker potential to discriminate different diagnostic categories based on logistic regression models. The optimal cut-off value corresponded to 5% of cells exhibiting strong (2+) KRT17 positive staining. This diagnostic property cut-off value (KRT17-5) was then applied to the entire dataset (n=114; 34 non-cancer specimens, 80 cancer specimens) and evaluated for significant differences related to detecting cancer versus non-cancer diagnoses using a chi-square test with odds ratio calculated.

Statistical Analysis: Examining the Relationship Between KRT17 and Survival.

To examine the relationship between KRT17 and survival, one cancer specimen per patient was analyzed. For example, if a subject had both primary and recurrent tumor specimens examined (n=1), the recurrent specimen(s) was removed from the cohort. If a patient had two primary specimens from the same time point (n=1), the patient's KRT17 for both primary specimen sample values were averaged. All non-cancerous specimens were excluded (n=34), for a final sample of n=78.

Initially, patient risk and tumor characteristics (i.e., age, HPV status, non-surgical treatments, stage, tumor location, and KRT17 as a continuous measurement) were compared based on the dichotomous survival variable (i.e., dead versus alive at end of study period) using the Wilcoxon rank-sum test, Fischer Exact test, or Chi-square test. For the time-to-death study endpoint, univariate survival analyses for all patient and tumor characteristics were performed using Kaplan-Meier or Cox proportional hazards models; multivariable analyses were performed including any variables reaching a univariate significance of p≦0.1. A prognostic cut-off point for KRT17 was then identified at strong KRT17 staining in at least 85% (KRT17-85), both graphically and by finding the optimal positive likelihood ratio. This KRT17-85 threshold was then incorporated into both the univariate and the multivariable survival models. Finally, a sensitivity analysis was performed to evaluate the variability in KRT17 thresholds across tumor location (e.g., tonsillar, laryngeal, and tongue) to assure that KRT17's use as a biomarker for prognosis of patient outcome and survival was robust. After calculating the positive likelihood ratios using the KRT17 85% cut off value, prognostic analyses were run for each subgroup of tumors by site. While tongue cancers performed well at the 85% cut-off value (LR=6.67, p<0.05), the optimal cut-off value for laryngeal and tonsillar carcinomas was 20-30% of cells in a sample exhibiting strong (2+) KRT17 staining.

In order to identify obvious divisions in the distribution, laryngeal and tonsillar carcinomas, were graphically analyzed. Tonsillar and tongue cancers showed different results; for tongue cancers Kaplan-Meier curves were created using the 85% strong KRT17 staining cut off—(+/−5% on either side, i.e., 80% and 90%, were also tested to ensure that the 85% was the correct determination), and 20% strong KRT17 staining cut off for tonsillar cancers (20%, 25%, and 30% were also tested, with the best fit occurring at 20%).

The diagnostic endpoint was the presence or absence of cancer, while dual prognostic endpoints were evaluated including, 1) vital status (i.e., death versus survival status as of Aug. 1, 2013); and 2) time-to-death, all patient deaths were assessed as of a common date (Aug. 1, 2013). Statistical significance was set at 0.05 and analysis was carried out using SAS 9.3 (SAS Institute, Inc., Cary, N.C., USA). All data are expressed as mean±SEM. One-way analysis of variance (ANOVA) was conducted for multiple group comparisons and the students' t-test was used when two groups were compared. Values were considered significantly different if p<0.05.

### Example 2

### Cytokeratin 17 is Overexpressed in Lingual, Tonsillar, and Laryngeal SCCs

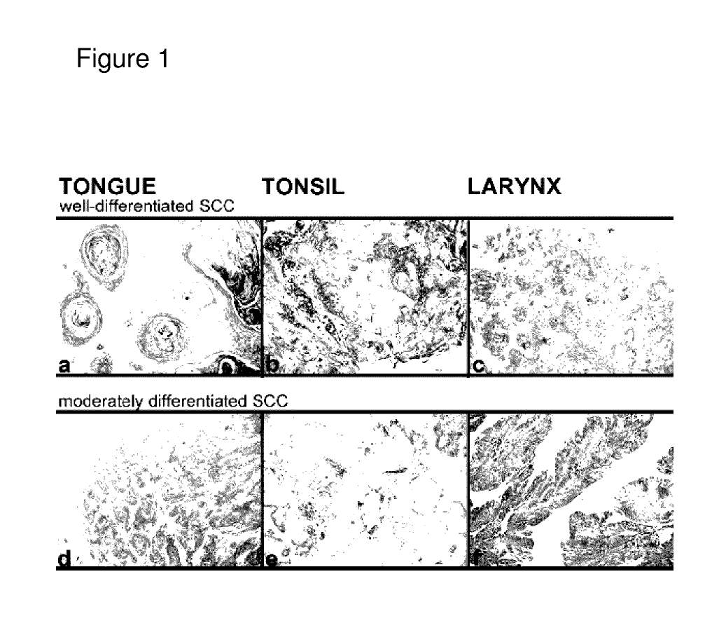

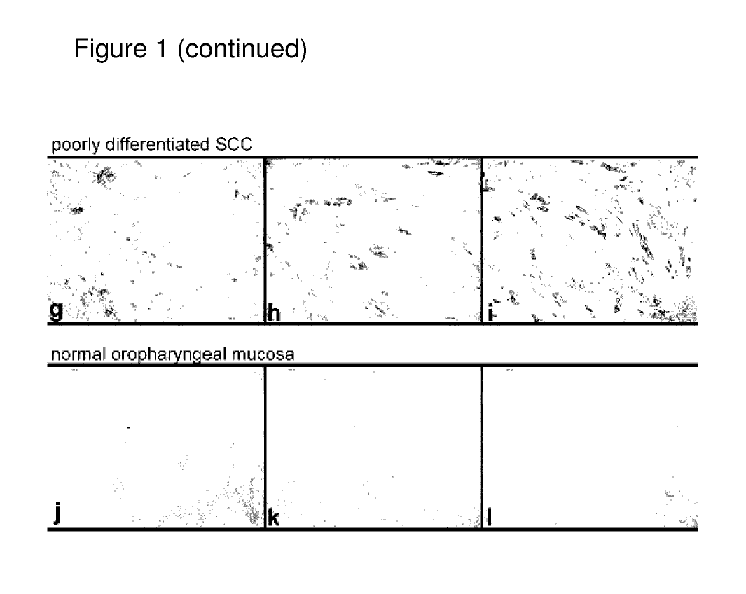

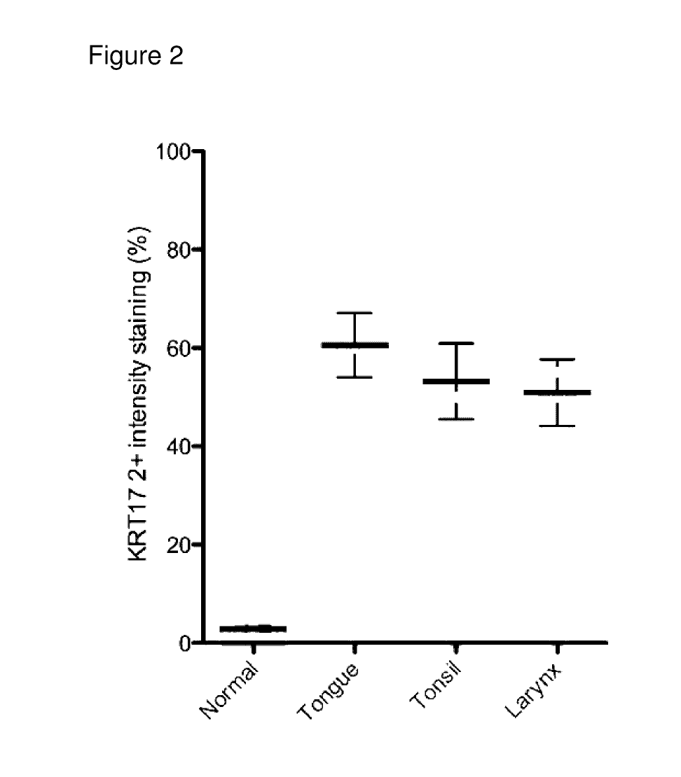

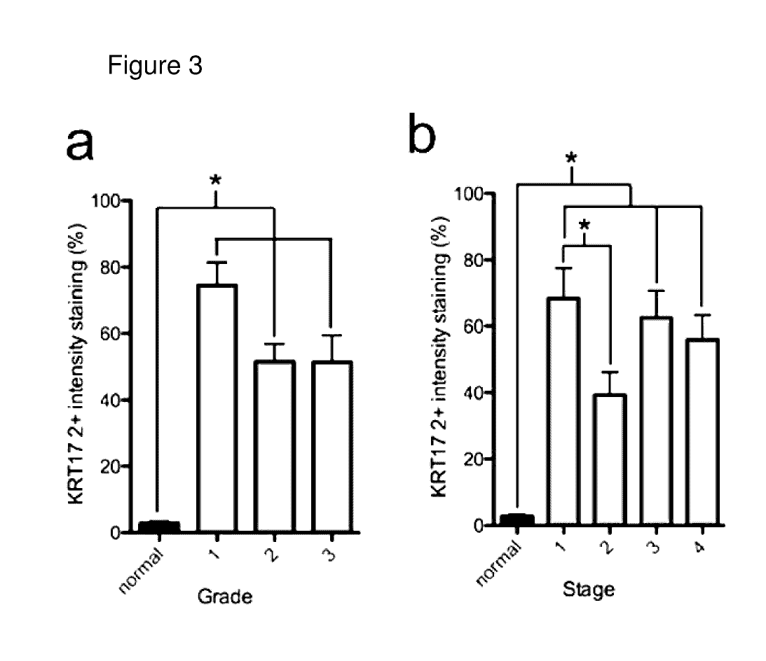

Cytoplasmic staining for KRT17 was detected in 78 of 78 (100%) HNSCC tissues specimens (FIG. 1a-i). The proportion of cells with strong (2+) staining ranged from 0 to 100% in the HNSCCs, with a mean 2+KRT17 expression of 60.6±6.6% in lingual, 53.2±7.7% in tonsillar, and 50.9±6.8% in laryngeal carcinomas (FIG. 2). When tumors were grouped by grade, KRT17 expression was reduced in poorly differentiated SCCs (51.5%±8.0%) and moderately differentiated SCCs (51.6%±5.3%) when compared to in well-differentiated SCCs (74.6%±6.9%) (FIG. 3a). When tumors were grouped by stage, KRT17 expression was significantly increased in all stages of HNSCC when compared to normal squamous mucosa (i.e., control samples).

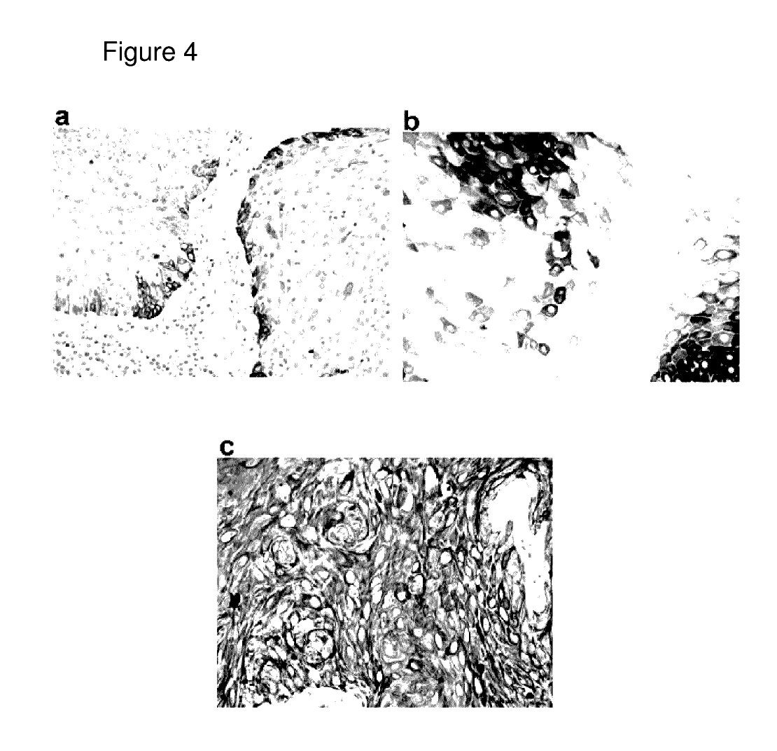

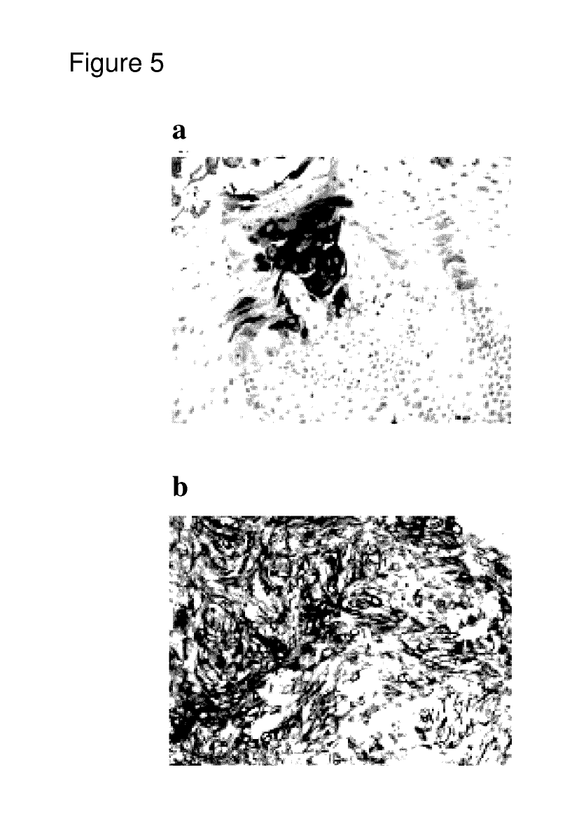

Within the SCCs, several patterns of KRT17 distribution were observed. For example, certain samples exhibited staining primarily at the periphery of invasive nests of tumor cells (FIG. 4a). Other samples showed scattered positive cells throughout the tumor tissue in a checkerboard-like pattern (FIG. 4b), and other samples showed uniform KRT17 staining throughout the tumor (FIG. 4c). Benign surface squamous mucosa was negative for KRT17 (FIG. 1j-l), but staining was often detected in both low-grade squamous intraepithelial lesions (LSIL) (FIG. 5a) and in high-grade squamous intraepithelial lesions (HSIL) (FIG. 5b).

### Example 3

### Cytokeratin 17 Expression is Consistent Between the Primary Tumor and Nodal Metastases

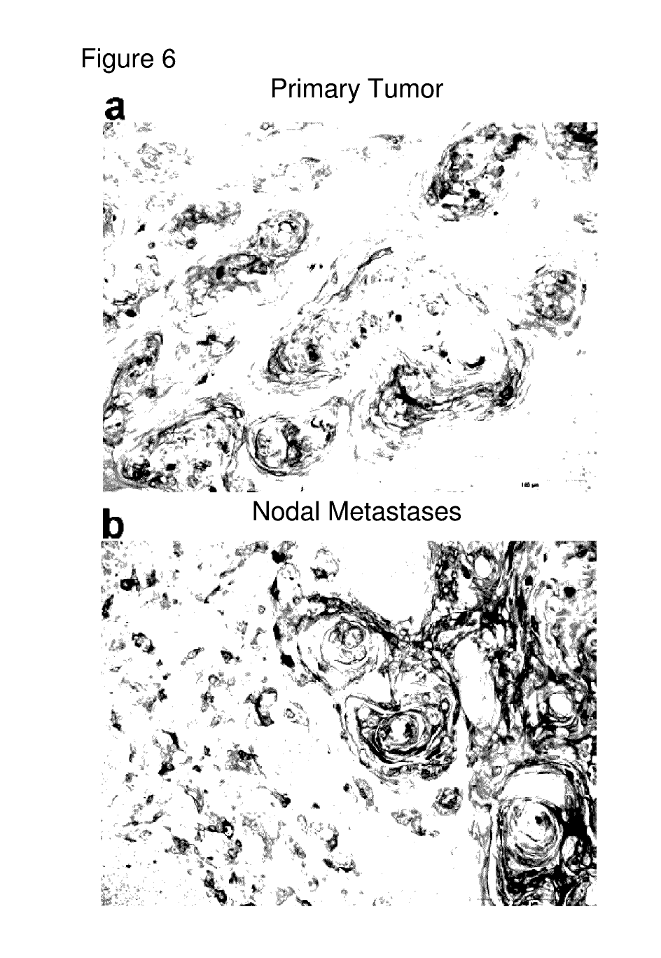

KRT17 expression in six representative cases was compared between the primary tumor and the concurrently diagnosed lymph node metastases. In five of the six cases, the intensity of staining between primary and metastatic tumor sites was consistent (FIG. 6a, b), i.e., primary tumors with high (2+) KRT17 staining had nodal metastases with high (2+) KRT17 staining.

### Example 4

### KRT17 Expression was Unrelated to HPV Status

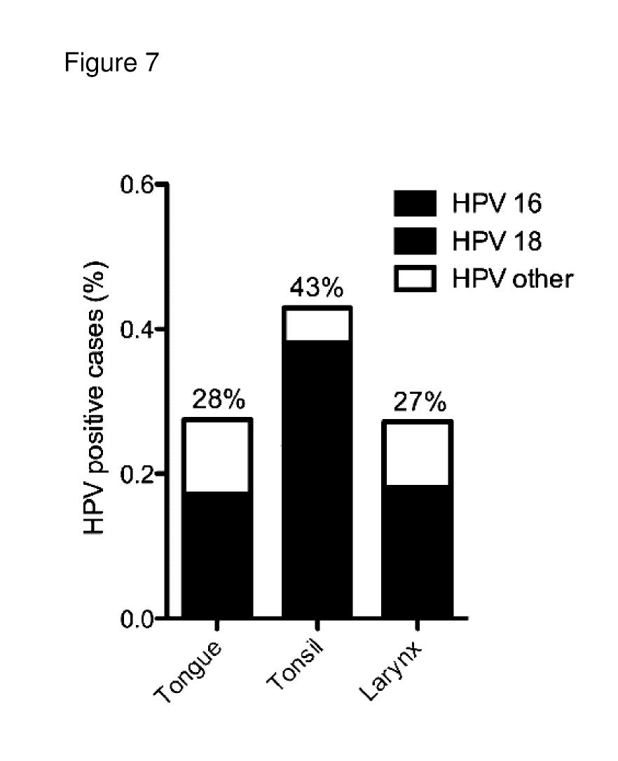

HPV DNA was detected in 27.6% of lingual SCCs (n=8/29), 42.9% of tonsillar SCCs (n=9/21), and 27.3% of laryngeal SCCs (n=6/22) (FIG. 7). Among the samples that tested positive, 3.4% of tongue cases were identified as HPV16 positive and 13.8% of tongue samples were identified as HPV18 positive. Further, 23.8% of tonsillar samples were identified as HPV16 positive and 14.3% of tonsillar samples were identified as HPV18 positive. Lastly, 4.5% of laryngeal samples were identified as HPV16 positive and 13.6% of laryngeal cases were identified as HPV18 positive. Notably, no co-infection by HPV 16 or 18 was detected in any of the samples analyzed. Univariate analysis determined that there was no significant correlation between KRT17 expression and HPV status (p=0.5391).

### Example 5

### KRT17 Thresholds Identified

Two thresholds for KRT17 were established a-priori to optimize the positive likelihood values for: 1) diagnostic threshold for KRT17 staining is strong (i.e., 2+KRT17 staining) KRT17 staining in 5% (KRT17-5); and 2) prognostic threshold for KRT17 staining is strong (i.e., 2+KRT17 staining) KRT17 staining in ≧85% (KRT17-85). These two thresholds were used in all analyses performed.

### Example 6

### KRT17 Diagnostic Test Performance

Based on the KRT17-5 diagnostic threshold, the KRT17-5 biomarker had sensitivity of 85% and specificity of 94% with a positive likelihood ratio of 14.5 to detect cancer cases (n=80) from non-cancer (n=34) cases (p<0.0001). Thus, overall very good diagnostic test performance for KRT17-5 was demonstrated. The likelihood ration was carried out using the formula:

$$LR += \frac{sensitivity}{1 - specificity} ,$$

wherein LR+ is the likelihood of a diagnostic test to accurately detect a positive disease occurrence, i.e., the probability that a subject who has the disease tests positive divided by the probability of a subject who does not have the disease testing positive. Sensitivity is the number of true positives analyzed (i.e., test positive and actually develop a disease pathology), and specificity is the number of samples that test positive but do not develop disease.

### Example 7

### KRT17 Prognostic Test Performance

To evaluate for prognosis, two study endpoints were evaluated: 1) death versus survival during the study follow-up period; and 2) time-to-death endpoint (censored for patients surviving to the end of study follow-up period). Across all tumor types, the patient risk characteristics and treatments, as well as the mortality rates (i.e., deaths versus survivals during the follow-up study period), are described in Table 1. As noted, the median KRT17 values for the patients that died versus survived during the study follow-up period was different (p=0.05). Thus, a time-to-death analysis was performed.

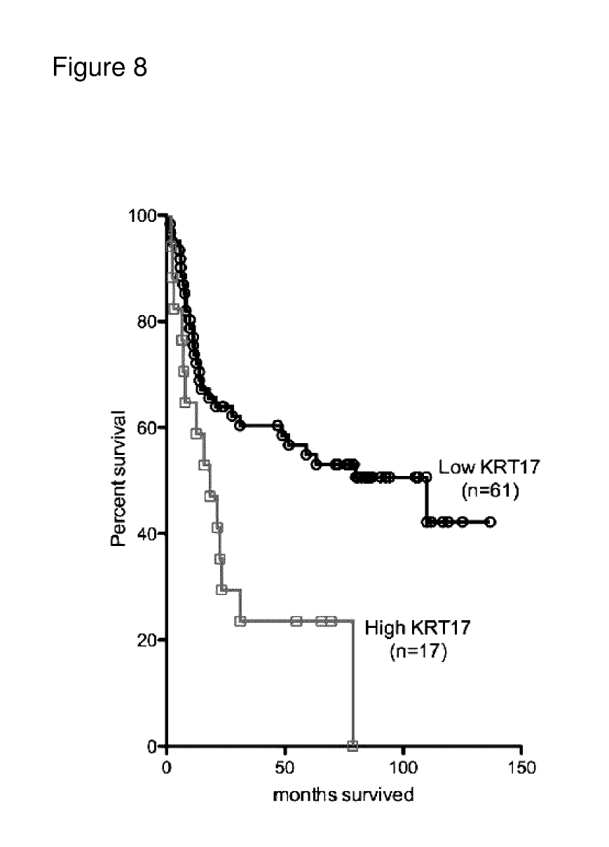

The Kaplan Meier analysis identified that KRT17-85 was a very strong predictor of survival (p=0.006) (FIG. 8). For the Cox Proportional Hazards model, patient-specific risk, specimen-specific assessments, and treatments were added as covariates (e.g., age, chemotherapy, radiation therapy, and tumor stage) along with the KRT17-85 findings; with KRT17-85 being a statistically significant predictor of multivariable survival (p=0.0124).

### Example 8

### Threshold Sensitivity Analysis by Tumor Location

To establish if KRT17 also had a prognostic effect across each of the three primary anatomic sites, tumor location-specific KRT17 thresholds were examined using the same method of analysis. The respective optimal thresholds for tonsillar and tongue SCCs were 20% (p=0.02) and 85% (p=0.006), respectively. However, there was not an optimal threshold that could be identified for laryngeal tumors (p=0.46). Although there was substantial variability in the optimal KRT17 threshold identified across tumor types, the KRT17-85 performed optimally across all tumor locations for the entire study population.

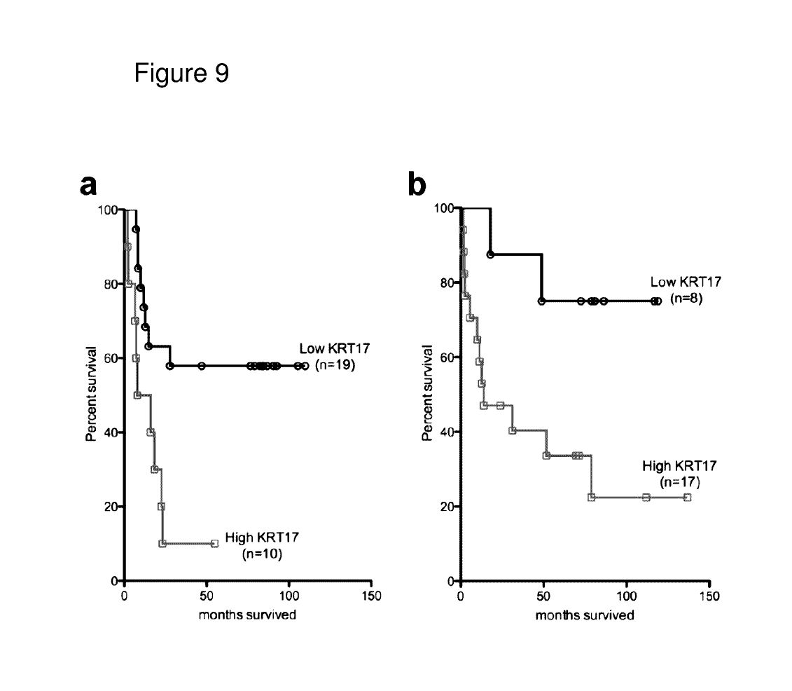

Using the Cox proportional hazards model (controlling for stage, radiation, chemotherapy, age, and HPV status), KRT17 site-specific threshold status and survival were correlated in lingual (hazard ratio=3.794, p=0.0061) (FIG. 9a) and tonsillar SCCs (hazard ratio=5.921, p=0.0235) (FIG. 9b).

1. A method of diagnosing a mammalian subject with head and neck squamous cell carcinoma (HNSCC) comprising
(a) obtaining a sample from said subject; and
(b)
detecting the level of KRT17 expression in said sample, wherein said detecting comprises contacting said sample with an mouse monoclonal antibody that binds KRT17 protein and determining the level of KRT17 bound to said mouse monoclonal antibody, and wherein KRT17 expression in at least 5% of cells in said sample indicates the presence of HSNCC in said subject.

2. The method of claim 1, further comprising processing the sample.

3. The method of claim 2, wherein said processing the sample comprises dissecting the sample to isolate cells, fixing the dissected sample with formalin, and embedding the formalin-fixed sample in paraffin.

4. The method of claim 1, wherein said sample is selected from the group consisting of tissue, biopsy, lymph node, or a combination thereof.

5. The method of claim 4, wherein said sample is a tumor biopsy sample or formalin-fixed paraffin-embedded tissue sample.

6. The method of claim 1, wherein said detecting the level of KRT17 in the sample is carried out by a process selected from the group consisting of individual tissue microarray with immunohistochemistry, immunofluorescent assay, Western blotting and immunohistochemical staining with microscopy.

7. The method of claim 1, wherein said detecting the level of KRT17 in the sample is carried out by immunohistochemistry, and wherein said mouse monoclonal antibody is [E3] anti-human KRT17 antibody.

8. The method of claim 1, wherein said HNSCC is selected from the group consisting of laryngeal squamous cell carcinoma, lingual squamous cell carcinoma and tonsillar squamous cell carcinoma.

9. The method of claim 1, further comprising comparing the detected level of KRT17 expression in the sample to that of a control sample, wherein the control sample is a non-cancerous tissue obtained from the subject, or a sample from healthy or non-cancerous tissue from other subjects.

10. The methods of claim 9, wherein said healthy tissue is selected from the group consisting of oropharyngeal mucosa and benign squamous mucosa.

11. The method of claim 1, wherein KRT17 expression in at least 65% of cells in said sample indicates the presence of HSNCC in said subject.

12. A method of determining the likelihood of survival of a subject having HNSCC comprising detecting the level of KRT17 expression in a sample obtained from the subject, wherein said detecting comprises contacting said sample with an mouse monoclonal antibody that binds KRT17 protein and determining the level of KRT17 protein bound to said mouse monoclonal antibody, and wherein KRT17 expression in at least 85% of cells in the sample identifies the subject as having a reduced likelihood of surviving at least 120 months post-diagnosis with HNSCC.

13. The method of claim 12, wherein said detecting the level of KRT17 expression is determined by immunohistochemical staining of said sample, and said mouse monoclonal antibody is [E3] anti-human KRT17 antibody.

14. The method of claim 2, wherein said reduced likelihood of survival is less than a 50% chance of surviving at least 120 months post-diagnosis with HNSCC.

15. The method of claim 2, wherein said reduced likelihood of survival is less than about a 25% chance of surviving at least 120 months post-diagnosis with HNSCC.

16. The method of claim 2, wherein said HNSCC is lingual squamous cell carcinoma, and wherein said reduced likelihood of survival is less than about a 25% chance of surviving at least 120 months post-diagnosis with HNSCC.

17. The method of claim 12, wherein said HNSCC is selected from the group consisting of laryngeal squamous cell carcinoma, lingual squamous cell carcinoma and tonsillar squamous cell carcinoma.

18.-19. (canceled)

20. The method of claim 1, wherein said KRT17 expression in between 60% and 77% of cells in said sample indicates the presence of Stage 1 HSNCC in said subject.

21. The method of claim 1, wherein said KRT17 expression in between 32% and 48% of cells in said sample indicates the presence of Stage 2 or Stage 3 HSNCC in said subject.

22. The method of claim 1, wherein said KRT17 expression in between 49% and 59% of cells in said sample indicates the presence of a Stage 4 HSNCC in said subject.


SEQUENCE LISTING (6 sequences)

SEQ ID NO: 1 - DNA, 24 bp, Artificial Sequence
  HPV Type 16 forward primer
cgcacaaaacgtgcatcggctacc

SEQ ID NO: 2 - DNA, 23 bp, Artificial Sequence
  HPV Type 16 reverse primer
tgggaggccttgttcccaatgga

SEQ ID NO: 3 - DNA, 26 bp, Artificial Sequence
  HPV Type 18 forward primer
aacagtccattaggggagcggctgga

SEQ ID NO: 4 - DNA, 21 bp, Artificial Sequence
  HPV Type 18 reverse primer
tgccgccatgttcgccatttg

SEQ ID NO: 5 - DNA, 20 bp, Artificial Sequence
  Beta-globin gene forward primer
caacttcatccacgttcacc

SEQ ID NO: 6 - DNA, 20 bp, Artificial Sequence
  Beta-globin gene reverse primer
gaagagccaaggacaggtac